(19) United States
(12) Patent Application Publication
(10) Pub. No.: US 2018/0103841 A1
(43) Pub. Date: Apr. 19, 2018

(54) SYSTEMS FOR VISUAL FIELD TESTING

(71) Applicants: Carl Zeiss Meditec, Inc., Dublin, CA (US); Carl Zeiss Meditec AG, Jena (DE)

(72) Inventor: Robert SPROWL, Livermore, CA (US)

(21) Appl. No.: 15/568,183

(22) PCT Filed: Apr. 28, 2016
(86) PCT No.: PCT/EP2016/059552

§ 371 Date: Oct. 20, 2017

Related U.S. Application Data
(60) Provisional application No. 62/155,746, filed on May 1, 2015.

(51) Int. Cl. A61B 3/032; A61B 3/028; G02B 3/14; A61B 3/14

(52) U.S. Cl. CPC A61B 3/032; A61B 3/14; G02B 3/14; A61B 3/0285

(57) ABSTRACT

Systems for visual field testing are described. One example system for testing the visual function of a patient includes a display, optics, a variable focus lens, a response detection system, and a processor. The display generates visual stimuli for the visual function testing of the patient. The optics image the visual stimuli onto the retina of the patient's eye. The variable focus lens is placed at a plane conjugate to the pupil of the eye for correcting the refractive error of the eye without impacting a field of view of the system. The response detection system collects data on the patient's perception of the visual stimuli. The processor receives a refractive error value of the patient and in response adjusts the variable focus lens to compensate for the refractive error of the patient.

### FIELD OF THE INVENTION

The present application concerns the refractive correction of patients without altering or obstructing a field of view, and/or varying the spacing of one or more elements of an optical system. In particular, the invention discussed in the present application describes placing a variable focus lens such as a liquid lens for refraction correction and/or using a microlens array for expanding the exit pupil in an optical system without affecting the field of view or image focus.

### BACKGROUND

A visual field analyzer or perimeter sends a stimulus of varying size and brightness to different parts of the retina. When the patient detects the stimulus at a particular location, a button is clicked. In this way, a map of the visual field is created. A traditional perimeter like the HFA sold by Carl Zeiss Meditec (Dublin, Calif.) projects the stimulus onto a bowl (see for example, U.S. Pat. No. 5,323,194, the contents of which are hereby incorporated by reference). The stimulus scatters from the bowl surface and is detected by the patient. A perimeter can also be made in which the patient views the stimulus as a virtual image. Any virtual image display with a large enough field of view can be used for perimetry testing as long as the brightness, dynamic range, and field of view are sufficient. One such virtual image field analyzer is the Zeiss Matrix.

One way to make a virtual visual field analyzer is to use a microdisplay in combination with lenses that form an exit pupil (see for example US Patent Publication No. 2010/0315594, the contents of which are hereby incorporated by reference). When the eye is placed at the exit pupil, the image of the microdisplay is formed on the retina. Some of the examples of microdisplays are LCD, LCOS, OLED, and DLP. Lenses are used to image the microdisplay onto the retina with the desired field of view, eye relief, and exit pupil size. FIG. 1 shows the basic components of one such virtual visual field system 100. As depicted, the system 100 includes a microdisplay 104 for creating a stimulus shape, and controlling position and intensity of the stimulus that is imaged to the eye 108 using imaging optics, such as an ocular lens 106 and other lenses following the microlens array 104. The system 100 includes a light source, such as a LED 102 for illuminating the microdisplay 104. Reference numeral 110 shows a plane conjugate to the exit pupil of the eye 108 and reference numeral 112 shows a plane conjugate to the retina. A response detection system (not shown) would be used to collect information on the subject's perception of the presented visual stimuli. Furthermore, a processor (not shown) would be used to process the information received from the response detection system and display or store results of the processed information thereof.

In perimetry, patient's glasses are removed because they may interfere with the stimulus due to the frame size or impact the test results in an unknown way due to the specific lens curvature. Instead, large aperture trial lenses are typically used so that the fixation and stimuli are in focus on the retina. Trial lenses can also be used in a visual field analyzer that uses a virtual image by simply placing the trial lens between the eye and the ocular lens. U.S. Pat. No. 8,668,338 hereby incorporated by reference describes replacing the standard trial lens in front of the patient's eye with variable focus lens such as a liquid lens. However, placing a lens directly in front of a patient's eye may not be an ideal location for refraction correction in virtual image based visual field testing systems because it may alter the angle of the stimulus. Other methods to correct for refractive error may include moving the retinal conjugate relative to the ocular lens (eyepiece) by one or more of 1) moving the ocular lens, 2) moving the instrument relative to the ocular lens, or 3) by adjusting lenses relative to each other. These methods require changing the element spacing along the optical axis. Typically a range of +/−20 Diopters of spherical power is required to cover the entire population.

### SUMMARY

The present invention describes how a variable focus lens such as a liquid lens and a microlens or variable focus microlens array can be used to improve a virtual image based vision field analyzer. According to one aspect of the subject matter described in the present application, a system for testing the visual function of a patient includes a display for generating visual stimuli; optics for imaging the visual stimuli onto the retina of the patient's eye; a variable focus lens placed at a plane conjugate to the pupil of the eye for correcting the refractive error of the eye without impacting a field of view of the system; a response detection system for collecting data on the patient's perception of the visual stimuli; and a processor for receiving a refractive error value of the patient and in response adjusting the variable focus lens to compensate for the refractive error of the patient.

According to another aspect of the subject matter described in the present application, a system for testing the visual function of a patient includes a display for generating visual stimuli; optics for imaging the visual stimuli onto the retina of the patient's eye; a microlens array placed at a plane conjugate to the retina for expanding exit pupil size without changing the focus; and a response detection system for collecting data on the patient's perception of the visual stimuli.

According to yet another aspect of the subject matter described in the present application, a system for testing the visual function of a patient includes a display for generating visual stimuli; optics for imaging the visual stimuli onto the retina of the patient's eye; a variable focus lens placed at a plane conjugate to a scan mirror or at an image of a scan mirror for correcting the refractive error of the eye without impacting a field of view of the system; a response detection system for collecting data on the patient's perception of the visual stimuli; and a processor for receiving a refractive error value of the patient and in response adjusting the variable focus lens to compensate for the refractive error of the patient.

One or more of these aspects may each optionally include one or more of the following features.

For instance, the features may include that the variable focus lens is a liquid lens; that the display is a microdisplay; that the variable focus lens adds or subtracts optical power to the system without changing the field of view or an exit pupil position; that the variable focus lens refocuses the visual stimuli while keeping the stimuli size constant; that the microlens array expands the numeral aperture without changing the focus or field of view; that the microlens array includes a plurality of microlenses; that the size and focal length of each lenslet in the microlens array is chosen based on one or more of the numerical aperture of the light entering the micro lens array, the required output numeral aperture, and the system resolution; that the micro lens array is a variable power micro lens array for automatically adjusting the exit pupil size according to the size of the patient's eye pupil; and that the variable power microlens array includes an array of tiny liquid lenses.

The present invention is advantageous in a number of respects. By way of example and not limitation, (1) the invention enables optical power to be added or subtracted to an optical system without changing the field of view, image size, or exit pupil position; (2) by placing the variable focus lens such as a liquid lens at a pupil, instead of another location, components before the pupil do not have to grow to accommodate a diverging beam; (3) the stimulus and fixation can be brought into focus much faster than by moving optical elements relative to each other or moving an aperture relative to a lens or moving an intermediate image plane.

It should be understood that the invention discussed herein is not limited to visual field analyzers/testers/systems and/or perimeters, and can be used in conjunction with any system that creates an image on the retina and makes use of a variable focus lens.

The features and advantages described herein are not all-inclusive and many additional features and advantages will be apparent to one of ordinary skill in the art in view of the figures and description. Moreover, it should be noted that the language used in the specification has been principally selected for readability and instructional purposes and not to limit the scope of the inventive subject matter.

### BRIEF DESCRIPTION OF THE DRAWINGS

FIG. 1 illustrates some basic components or elements of an example virtual visual field analyzer.

FIG. 2 illustrates a virtual image based vision field instrument according to one embodiment of the present invention.

FIG. 3 illustrates a liquid lens that could be used in one embodiment of the present invention.

FIGS. 4A and 4B are two exemplary layouts illustrating location of a liquid lens in an optical system. In particular, FIG. 4A is a layout showing liquid lens location at a plane conjugate to the stop aperture and exit pupil. FIG. 4B is a layout showing liquid lens location near a scan mirror or at an image of the scan mirror.

FIG. 4C shows an application of the layout of FIG. 4A to an example fundus camera system.

FIG. 5 shows the layout of FIG. 4A with only chief rays depicted.

FIG. 6 is an example illustrating how a liquid lens located at a plane conjugate to the exit pupil can shift the focus without affecting field of view or mechanically moving components along the optical axis.

FIG. 7 is an example configuration of a visual field analyzer having a micro lens array placed at the retinal conjugate for expanding the numeral aperture (NA) and exit pupil size.

FIG. 8 shows an exemplary way of using microlenses for NA expansion and a liquid lens for refraction correction in the same instrument/optical system without axial adjustment.

FIG. 9 is an example layout depicting locations of a microlens array and a liquid lens in the system of FIG. 1.

### DETAILED DESCRIPTION

All patent and non-patent references cited within this specification are herein incorporated by reference in their entirety to the same extent as if the disclosure of each individual patent and non-patient reference was specifically and individually indicated to be incorporated by reference in its entirely.

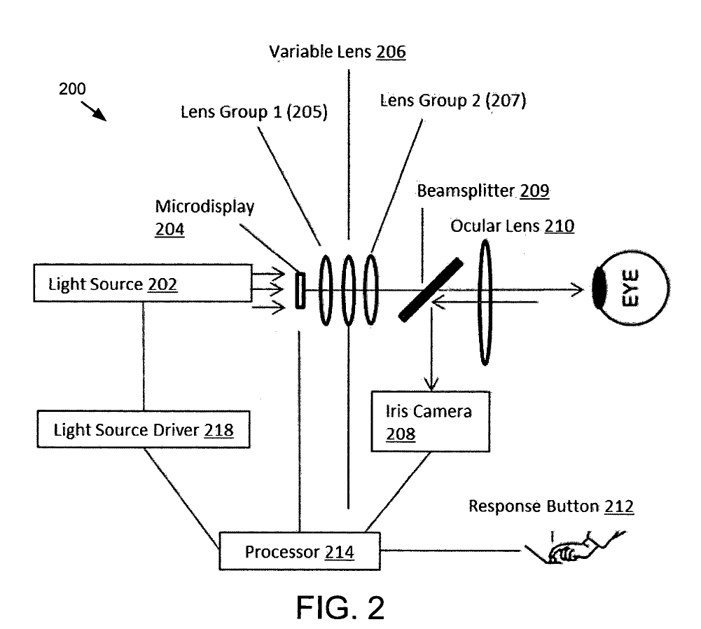

FIG. 2 illustrates a virtual image based visual field instrument 200 according to one embodiment of the present invention. As depicted, the instrument 200 includes a light source 202, a microdisplay 204, a lens group 1 (205), a variable lens 206, a lens group 2 (207), an optional iris camera 208, an ocular lens 210, a response button 212, a processor 214, a beamsplitter 209, and a light source driver 218. It should be noted that the instrument 200 is not in any way limited to the elements/components depicted in FIG. 2 and may include one or more additional components as needed to perform the invention discussed in the present application. Each component of the instrument 200 is discussed further below.

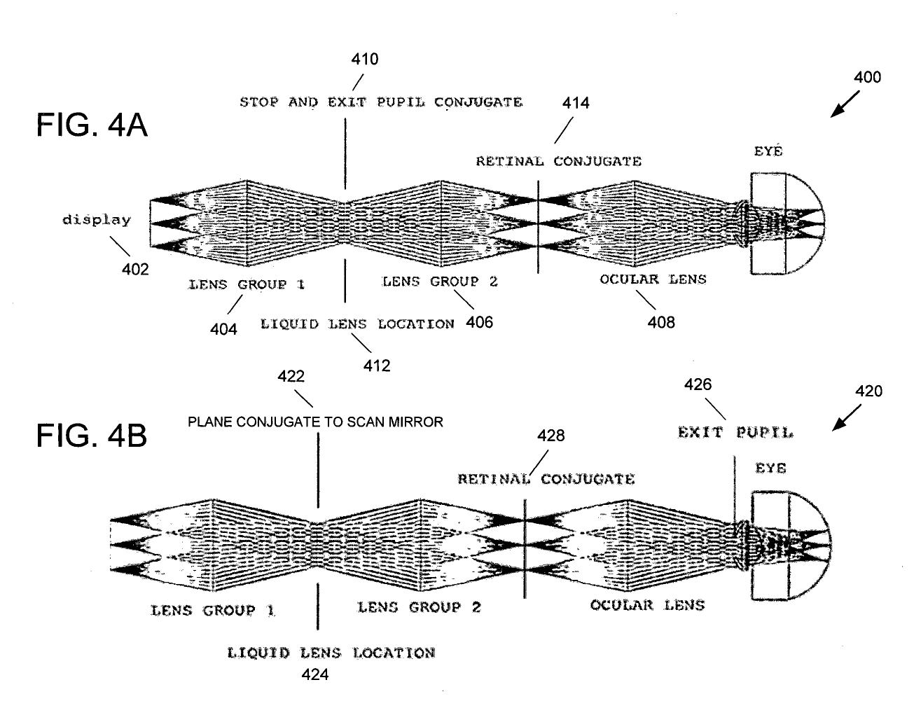

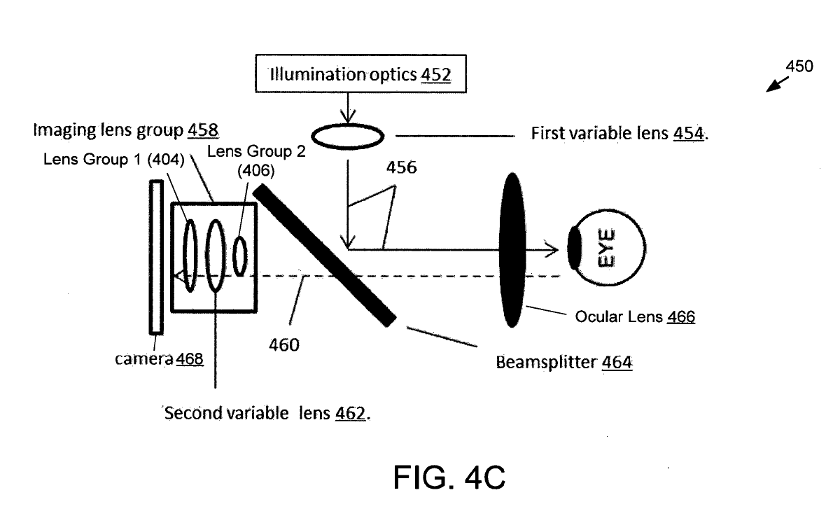

The light source 202 is used to illuminate the microdisplay 204. The light source 202 is controlled by the light source driver 218. A light source 202 can be, for example, a laser, a light-emitting diode (LED), a lightbulb, etc. The microdisplay 204 creates a stimulus image, which is projected onto the retina of the eye by the ocular lens 210. A patient indicates that a stimulus was seen by depressing the response button 212, such as a mechanical clicker. It should be noted that any sort of response or feedback mechanisms for providing patient's feedback that are standard and well known in the art could be used with the system 200. The response is recorded by the processor 214. The optional iris camera 208 is used to track patient's gaze and measure pupil diameter throughout the test. The beamsplitter 209 sends returning light or light reflected from the retina to the iris camera 208. The variable lens 206 such as a liquid lens is placed at a plane conjugate to the eye pupil and internal to the instrument 200, and is used to correct for refraction. As depicted, the variable lens 206 is placed in between the lens group 1 (205) and the lens group 2 (207). In some instances, the lens group 1 (205), the variable lens 206, and the lens group 2 (207) all together create an imaging lens group, such as the imaging lens group 458 shown in FIG. 4C. The processor 214 is operatively connected to the iris camera 208, response medium 212, and/or a variable lens 206 to receive patient's data (e.g., refractive error value of the patient) and in response adjust the variable lens 206 to compensate for the refractive error of the patient.

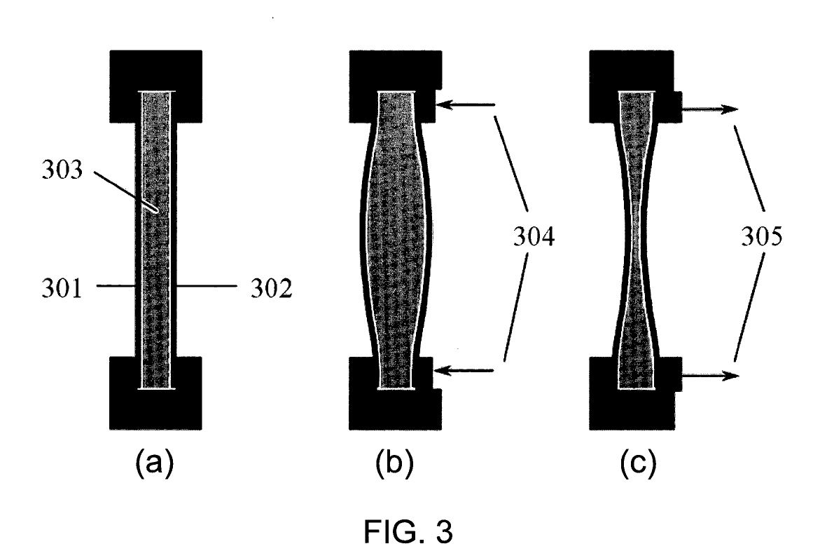

FIG. 3 illustrates an example liquid lens. Such a lens typically consists of one or two transparent and flexible membranes 301 and 302, encapsulating a volume of liquid 303 with a specific refractive index. A variety of liquid lenses have been described in the literature (see for example, U.S. Pat. No. 8,668,338, the contents of which are hereby incorporated by reference). Continuously adjustable refractive positive or negative powers of up to 25-50 diopters have been demonstrated (see for example, Ren, Hongwen, and Shin-Tson Wu. “Variable-focus liquid lens by changing aperture.” Applied Physics Letters 86.21 (2005): 211107). An actuator changes the distribution of the volume of the liquid, to adjust the refractive power of the lens as shown pictorially in FIGS. 3(b) and 3(c) creating convex and concave lenses respectively. In this case, pressure is applied or released to the periphery of the lens as indicated by arrows 304 and 305. The volume change can be accomplished either manually or automatically by the instrument, by tuning the radius of an annular sealing ring, or by squeezing or releasing the periphery of the lens or other method which changes the profile of the lens or volume of the liquid.

A variable lens such as a liquid lens placed at the system stop or another plane that is conjugate to the exit pupil can correct for refractive error without affecting the field of view, as shown for example in at least FIG. 2 and FIG. 4A. The liquid lens adds or subtracts optical power to the wavefront without affecting the chief ray angles that determine the system field of view. The liquid lens could include positive and negative spherical power as well as positive and negative cylindrical power and axis. An Alvarez lens which works by sliding two or more aspheric surfaces laterally relative to each other to change spherical power could also be used in place of the liquid lens with a similar result.

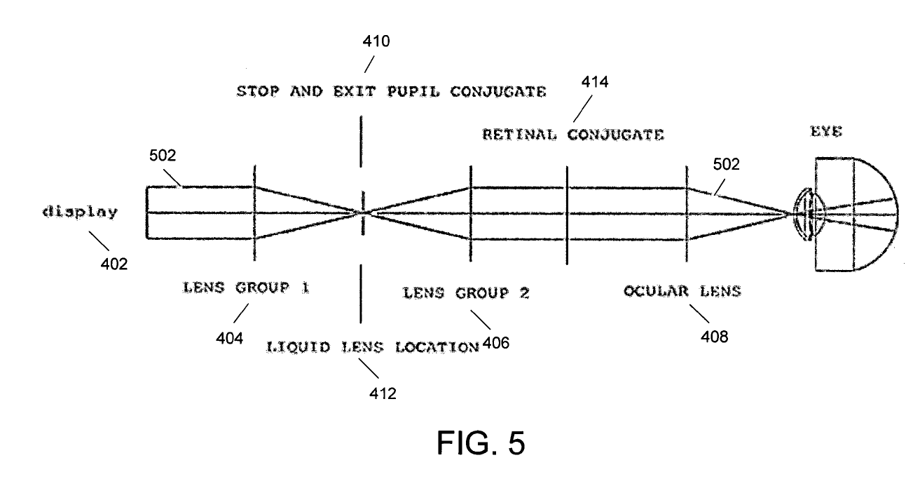

FIGS. 4A and 4B are two exemplary layouts 400 and 420, respectively, illustrating location of a liquid lens (indicated by reference numerals 412 and 424) in a system with 3 lenses or lens groups. These 3 lenses or lens groups are indicated by reference numerals 404, 406, and 408, respectively. The layout 400 shows the display 402, stop 410, and retinal conjugate 414. The exit pupil is an image of the stop 410 shown at the cornea in FIG. 4A. In this layout, the lenses (404, 406, and 408) are represented by the planes where the ray bending occurs. Each cone represents a different stimulus position. The liquid lens, as indicated by reference numeral 412, is placed at plane conjugate to the stop aperture and the exit pupil 410. One benefit of using the layout shown in FIG. 4A is that the magnification from the display 402 to the retinal conjugate 414 can be set to give the desired field of view for a given eye relief. Eye relief is the distance from the cornea to the final lens (e.g., lens 408) in a given system. By way of an example and with reference to FIG. 4B, if the cornea to lens distance is a constraint (for example 25 mm), lens group 1 (404) and lens group 2 (406) can be used to magnify the display 402 to the correction dimensions at the intermediate image plane. FIG. 5 shows only the chief rays (indicated by reference numeral 502) of the same layout/system shown in FIG. 4A. The rays 502 are unchanged as optical power is added or subtracted to the liquid lens. The chief rays 502 determine the field of view or image height.

The layout 420 in FIG. 4B shows the system of FIG. 4A with the stop aperture or exit pupil conjugate 410 replaced with a plane conjugate to a scan mirror or galvo 422. The exit pupil and retinal conjugate are now indicated by reference numerals 426 and 428, respectively. As depicted, the liquid lens is placed (as shown by reference numeral 424) near the scan mirror or at a plane that is an image of the scan mirror.

It should be noted that the layouts 400 and 420 shown in FIG. 4A and FIG. 4B, respectively, can be applied or used with a variety of other imaging instruments and not just the virtual visual field systems discussed in the present application. By way of example and not limitation, the layout 400 can be applied to an example fundus camera system 450 shown in FIG. 4C. As depicted, the system 450 includes illumination optics 452 that are placed at a plane conjugate to the retina. A first variable lens (e.g. liquid lens) 454 is used in the illumination path (indicated by reference numeral 456) and is placed at a plane conjugate to the eye pupil. The first variable lens 454 is used to focus an illuminating light onto the retina. The system 450 includes an imaging lens group 458 in the detection path (indicated by reference numeral 460) of the system. The imaging lens group 458 may consist of the lens group 1(404), the lens group 2 (406), and a second variable lens 462. The second variable lens 462 may be placed in between the lens group 1 (404) and the lens group 2 (406), and at a plane conjugate to the imaging system stop. The second variable lens 462 is used to focus the light returning from the eye onto the camera 468. The illumination path (456) and the detection path (460) are separated with the use of a beamsplitter 464. The system, as depicted, also includes an ocular lens 466 in the illumination 456 and the detection path 460.

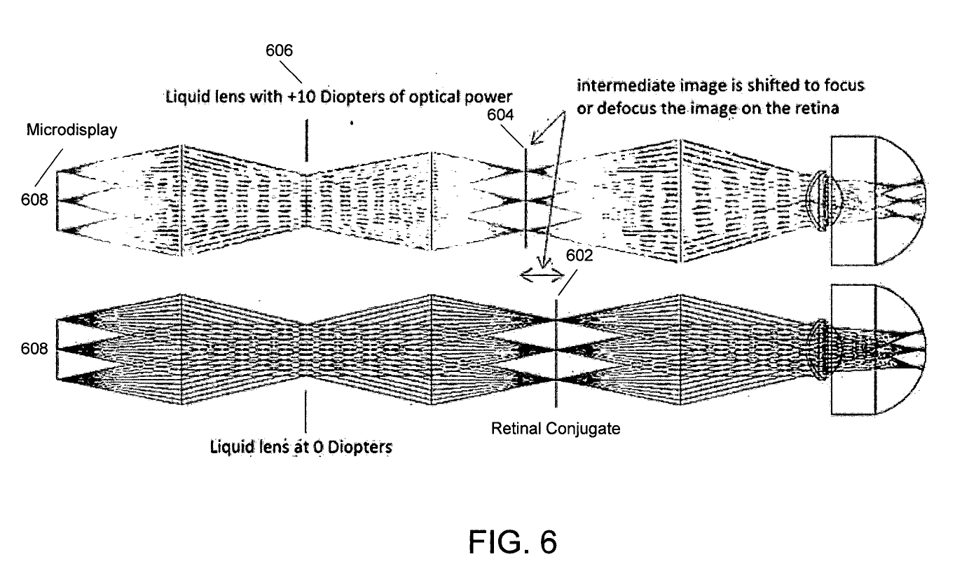

FIG. 6 is an example depicting how a liquid lens located at a plane conjugate to the exit pupil (606) can shift the focus without affecting the field of view or mechanically moving components along the optical axis. Specifically, this figure shows focus shift from 602 to 604 by just adding an optical power of +10 diopters to the liquid lens (indicated by reference numeral 606). Note that the liquid lens location is not changed due to the addition of this optical power. In this particular example, one to one magnification was used from the microdisplay (608) to the retinal conjugate (602 or 604), but it should be understood that this is not limiting and any magnification could be used.

A processor (e.g., the processor 214) operatively connected to the variable focus lens (e.g., the variable lens 206, see FIG. 2) would receive information on the refractive error of the particular eye being tested and in response would automatically adjust the variable focus lens to compensate for the refractive error of the patient. Today, most patients' refractive statuses reside in one or more patient databases and typically include the spherical and cylindrical parts of the refractive error and the angular orientation of the cylindrical part for both eyes of a patient. It would be advantageous to let the perimetry system automatically measure the refractive error for the patient to be tested, using an auto-refracting technique known to those skilled in the art, or retrieve it, by network or other means, from the patient database or record system. Alternatively, the operator can manually provide the perimeter with the refractive error values if the patient record is only available on paper. With the knowledge of the patient's refractive status, the instrument can then calculate the spherical equivalent power of the spherical lens(es) necessary to provide the patient with a well-focused view of the perimetric stimulus. The system can use an actuator, e.g., an electrical motor, to adjust the lens system to the correct power. It would be desirable to use a feedback system to ensure that the automatic spherical lens has the correct power and stays in calibration. If the instrument finds that the patient's refractive error is outside the range of the variable focus lens, the instrument can instruct the operator to add an additional refractive lens of specified power to the system to achieve the desired total power. This could also include adding cylindrical power to the system. The above described procedure would save a significant amount of time and reduce the risk of errors associated with preparing a patient for a visual field test.

If the refractive status of the patient is not known either by auto-refractive measurement or patient record or database, it could also be very advantageous to let the perimeter instrument use the variable focus lens to determine the refractive error of the patient. For example, a Snellen like chart for visual acuity could be displayed and the lens system for variable refraction error correction could be adjusted either by the virtual visual field tester or the operator until the patient can view the Snellen chart clearly.

When viewing images on virtual displays or near eye displays, the eye needs to be placed near the exit pupil in order to see the full image. The exit pupil should be larger than the eye pupil so that the eye can be displaced laterally without losing light and vignetting the image. The final size of the exit pupil is determined by the system magnification and the numerical aperture (NA) of the microdisplay. In FIG. 6, the numerical aperture is defined by the cone of rays in the first plane on the left that represents the microdisplay 608. In many types of microdisplays there is a tradeoff between uniformity and numerical aperture. As a result, numerical apertures of about 0.2 (full cone angle of 23°) are used. The low numerical aperture may result in an exit pupil that is too small to allow lateral displacements of the eye. Such a system would be very sensitive to patient alignment and motion.

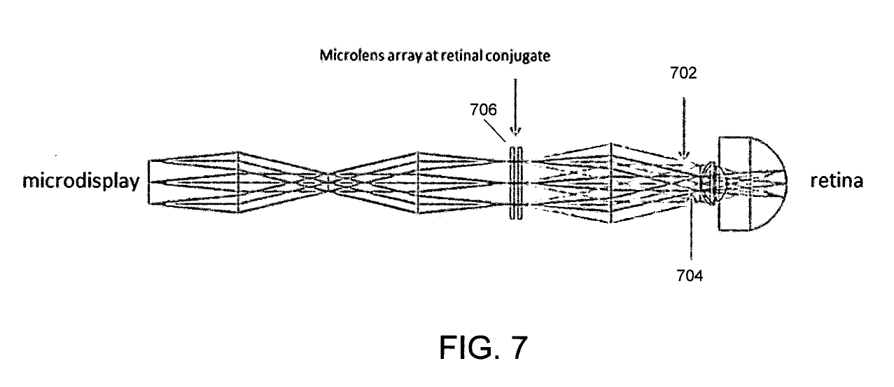

One solution to the above-discussed problem is to place a plurality of microlenses in a plane that is conjugate to the retina. The microlenses when placed at or near a plane conjugate to the retina have the effect of increasing the numerical aperture and the exit pupil size without affecting the location of the image plane or the image size. The size and focal length of each lenslet in the microlens array is chosen based on the numerical aperture of the light entering the microlens array, the required output numerical aperture and the system resolution. For example, a lenslet with a diameter of 0.5 mm and a focal length of 1 mm would have a numerical aperture of 0.25. The lenslets can have cylindrical, spherical, or aspherical curvature. The outer diameter of each lenslet is usually square or hexagonal to minimize gaps or obstructions between lenslets. A single array lens array can be used. Two lens arrays separated by an air gap can also be used. An example of how this can be used in a visual field analyzer is shown in FIG. 7 where a microlens array is placed at the retinal conjugate, as shown by reference numeral 706, to expand the numerical aperture and exit pupil size in a virtual visual field testing system. This is indicated by reference numerals 702 and 704. Reference numeral 702 represents the rays that show larger exit pupil after inserting the microlens array. Reference numeral 704 represents the rays that show smaller exit pupil without the microlens array. It is conceivable that one could use variable power microlenses, such as an array of tiny liquid lenses, to adjust the size of the exit pupil. This could be used to scale the exit pupil relative to the measured eye pupil for constant irradiance over a range of eye pupil sizes.

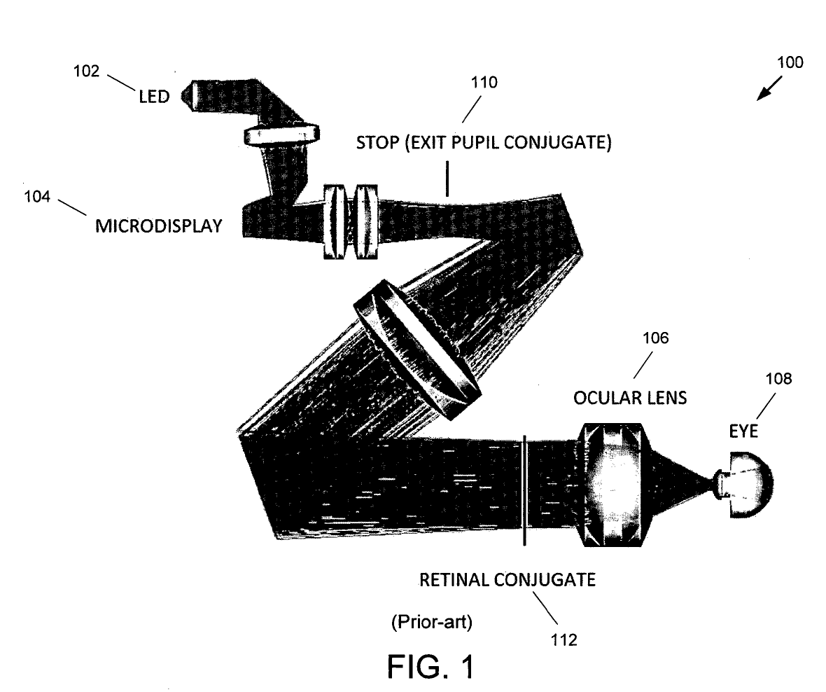

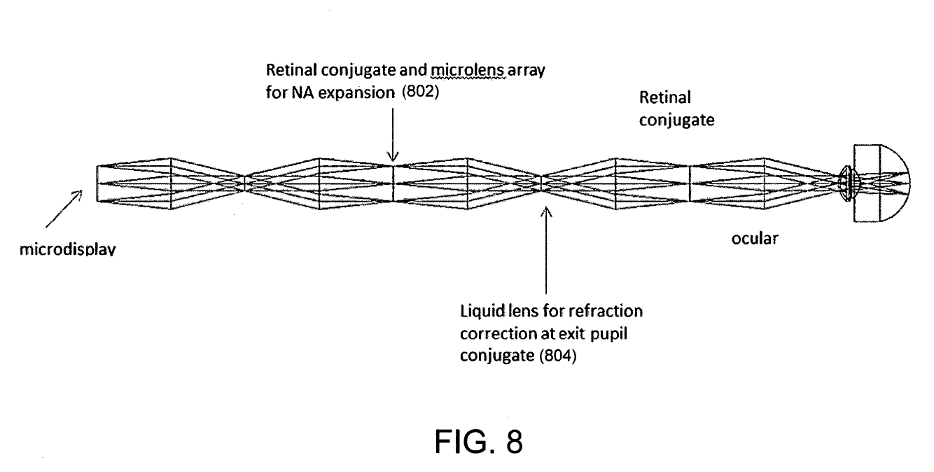

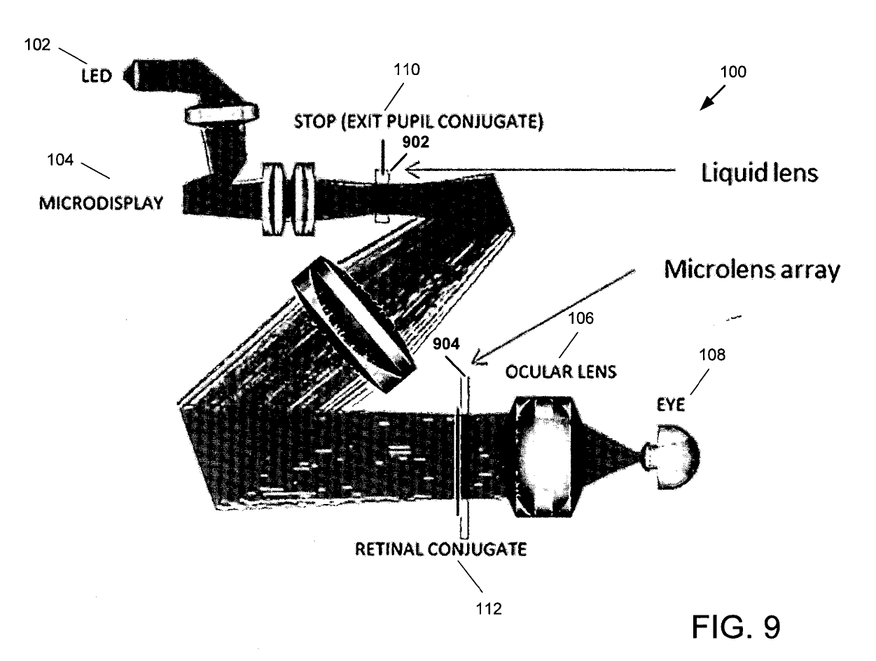

The exit pupil expansion with micro lens arrays and refraction correction with a liquid lens can be used individually within a system or combined in the same instrument. If the refraction correction is upstream from the microlens array there will be a shift in the retinal conjugate position and the microlens array will need to be shifted axially so that it is coincident with the retinal conjugate. If the system is arranged such that the refraction correction is done after the numerical aperture (NA) expansion, the components can be used together without moving any component axially. FIG. 8 shows one exemplary way to use microlenses for NA expansion and a liquid lens for refraction correction in the same instrument without axial adjustment. Specifically, FIG. 8 shows that the microlens array is placed at a plane conjugate to the retina (see reference numeral 802) for NA expansion and the liquid lens is placed at a plane conjugate to the exit pupil (see reference numeral 804) for refraction correction. FIG. 9 shows how the two elements i.e., the focus lens (liquid lens) 902 and microlens array 904 would fit into the overall system 100 shown in FIG. 1.

In the above description, for purposes of explanation, numerous specific details are set forth in order to provide a thorough understanding of the specification. It should be apparent, however, that the subject matter of the present application can be practiced without these specific details. It should be understood that the reference in the specification to “one embodiment”, “some embodiments”, or “an embodiment” means that a particular feature, structure, or characteristic described in connection with the embodiment is included in one or more embodiments of the description. The appearances of the phrase “in one embodiment” or “in some embodiments” in various places in the specification are not necessarily all referring to the same embodiment(s).

The foregoing description of the embodiments of the present subject matter has been presented for the purposes of illustration and description. It is not intended to be exhaustive or to limit the present embodiment of subject matter to the precise form disclosed. Many modifications and variations are possible in light of the above teaching. It is intended that the scope of the present embodiment of subject matter be limited not by this detailed description, but rather by the claims of this application. As will be understood by those familiar with the art, the present subject matter may be embodied in other specific forms without departing from the spirit or essential characteristics thereof. Furthermore, it should be understood that the modules, routines, features, attributes, methodologies and other aspects of the present subject matter can be implemented using hardware, firmware, software, or any combination of the three.

1. A system for testing the visual function of a patient, the system comprising:
a display for generating visual stimuli;
optics for imaging the visual stimuli onto the retina of the eye;
a variable focus lens placed at a plane conjugate to the pupil of the eye for correcting the refractive error of the eye without impacting a field of view of the system;
a response detection system for collecting data on the patient's perception of the visual stimuli; and
a processor operatively connected to the variable focus lens, said processor for receiving a refractive error value of the patient and in response adjusting the variable focus lens to compensate for the refractive error of the patient.

2. The system as recited in claim 1, in which the variable focus lens is a liquid lens.

3. The system as recited in claim 1, in which the display is a microdisplay.

4. The system as recited in claim 1, in which the variable focus lens adds or subtracts optical power to the system without changing the field of view or an exit pupil position.

5. The system as recited in claim 1, in which the variable focus lens refocuses the visual stimuli while keeping the stimuli size constant.

6. The system as recited in claim 1, further comprising an iris camera that measures patient's pupil size and tracks patient's gaze.

7. A system for testing the visual function of a patient, the system comprising:
a display for generating visual stimuli;
optics for imaging the visual stimuli onto the retina of the eye;
a microlens array placed at a plane conjugate to the retina for expanding exit pupil size without changing the focus; and
a response detection system for collecting data on the patient's perception of the visual stimuli.

8. The system as recited in claim 7, further comprising:
a variable focus lens placed at a plane conjugate to the pupil of the eye for correcting the refractive error of the eye without impacting a field of view of the system; and
a processor operatively connected to the variable focus lens, said processor for receiving a refractive error value of the patient and in response adjusting the variable focus lens to compensate for the refractive error of the patient.

9. The system as recited in claim 7, in which the microlens array further expands the numeral aperture without changing the focus or field of view.

10. The system as recited in claim 7, in which the microlens array includes a plurality of microlenses, wherein the size and focal length of each lenslet in the array is chosen based on one or more of the numeral aperture of the light entering the microlens array, the required output numeral aperture, and the system resolution.

11. The system as recited in claim 7, in which the display is a microdisplay.

12. The system as recited in claim 7 further comprising an iris camera that measures patient's pupil size and tracks patient's gaze.

13. The system as recited in claim 7, wherein the microlens array is a variable power microlens array for automatically adjusting the exit pupil size according to the size of the patient's eye pupil.

14. The system as recited in claim 13, in which the variable power microlens array includes an array of tiny liquid lenses.

15. A system for testing the visual function of a patient, the system comprising:
a display for generating visual stimuli;
optics for imaging the virtual stimuli onto the retina of the eye;
a variable focus lens placed at a plane conjugate to a scan mirror or at an image of a scan mirror for correcting the refractive error of the eye without impacting a field of view of the system;
a response detection system for collecting data on the patient's perception of the visual stimuli; and
a processor operatively connected to the variable focus lens, said processor for receiving a refractive error value of the patient and in response adjusting the variable focus lens to compensate for the refractive error of the patient.

16. The system as recited in claim 15, in which the variable focus lens is a liquid lens.

17. The system as recited in claim 15, in which the display is a microdisplay.

18. The system as recited in claim 15, in which the variable focus lens adds or subtracts optical power to the system without changing the field of view.
